(19) United States
(12) Patent Application Publication
(10) Pub. No.: US 2007/0074967 A1
(43) Pub. Date: Apr. 5, 2007

(54) Photodegradable protective group

(75) Inventors: Tetsuo Nagano, Tokyo (JP); Yasuteru Urano, Kanagawa (JP); Tomonori Kobayashi, Tokyo (JP); Noriyuki Suzuki, Chiba (JP)

Correspondence Address: GREENBLUM & BERNSTEIN, P.L.C., 1950 ROLAND CLARKE PLACE, RESTON, VA, 20191, US

(73) Assignee: The University of Tokyo, Tokyo (JP)

(21) Appl. No.: 11/366,464

(22) Filed: Mar. 3, 2006

Related U.S. Application Data
(60) Provisional application No. 60/658,172, filed on Mar. 4, 2005.

(51) Int. Cl. C07C 209/68; C07C 39/04; C07C 209/00

(52) U.S. Cl. 204/157.81; 564/388; 568/927; 568/584; 568/585; 204/157.9

(57) ABSTRACT

A photodegradable protective group comprising a benzyl group which is bound with a light capturing moiety that can absorb light of around 470 nm and having one or more nitro groups on the benzene ring.

### TECHNICAL FIELD

The present invention relates to a photodegradable protective group which is easily removed by irradiation of visible light.

### BACKGROUND ART

Photodegradable protective groups are protective groups that can be instantly deprotected by irradiation of light under a mild condition. Photodegradable protective groups are used for analysis of dynamic biological signal transmission as a so-called caged compound, and has also come to be used for organic syntheses such as solid phase DNA synthesis. As typical photodegradable protective groups, o-nitrobenzyl type protective groups are widely used (for example, α-carboxy-2-nitrobenzyl group, 1-(2-nitrophenyl)ethyl group, 4,5-dimethoxy-2-nitrobenzyl group, 1-(4,5-dimethoxy-2-nitrophenyl)ethyl group, 5-carboxymethoxy-2-nitrobenzyl group and the like, see, the catalog of Molecular Probes, Inc., “Handbook of Fluorescent Probes and Research Chemicals”, Ninth Edition, Chapter 17 (Caging groups and photolysis thereof)).

However, only a few kinds of photodegradable protective groups have been known so far. Moreover, they are required to be irradiated with light in the ultraviolet range for deprotection, and therefore they have a problem that they are not suitable for application to tests using live cells. Under the circumstances, development of protective groups is desired that can be removed by irradiation of light in the visible range and suitably applied to tests using live cells. Li et al. reported that molecules having coumarin and nitrobenzyl groups had high photodegradation efficiency (J. Am. Chem. Soc., 126, 4653-63 (2004)).

### DISCLOSURE OF THE INVENTION

An object of the present invention is to provide a protective group that can be removed by irradiation of light in the visible range and suitably applied for tests using live cells.

The inventors of the present invention conducted various researches to achieve the foregoing object, and as a result, they found that if a benzyl group having one or more nitro groups on the benzene ring and bound with a light capturing moiety which absorbs light in the visible range (especially light of around 470 nm) was used as a protective group of hydroxy group or the like, deprotection of the protective group successfully attained easily by irradiation of light in the visible range (especially light of around 470 nm). The present invention was accomplished on the basis of the aforementioned finding.

The present invention thus provides a photodegradable protective group comprising a benzyl group which is bound with a light capturing moiety that can absorb light of around 470 nm and having one or more nitro groups on the benzene ring. According to a preferred embodiment of this invention, there is provided the aforementioned protective group for protecting hydroxy group, amino group, carboxy group, or phosphate group.

The present invention also provides a method for removing a protective group comprising a benzyl group which is bound with a light capturing moiety that can absorb light of around 470 nm and having one or more nitro groups on the benzene ring, which comprises irradiating a compound having the protective group with light of around 470 nm, and provides a compound having the aforementioned protective group.

From another aspect, the present invention provides a reagent for introducing a photodegradable protective group, which is represented by the following formula (I): 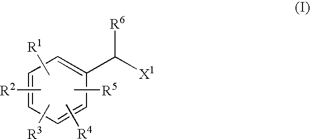
wherein X¹ represents a leaving group, R¹ represents hydrogen atom, or a light capturing moiety that can absorb light of around 470 nm, R², R³, R⁴, and R⁵ independently represent nitro group, hydrogen atom, or an arbitrary substituent substitutable on the benzene ring (when two to four of them represent substituents, they may be the same or different), provided that at least one of R², R³, R⁴, and R⁵ is nitro group, and R⁶ represents hydrogen atom, a light capturing moiety that can absorb light of around 470 nm, one or two monovalent substituents, or one divalent substituent (when R⁶ represents two substituents, they may be the same or different), provided that at least one light capturing moiety that can absorb light of around 470 nm exists as R¹ or R⁶. As a preferred embodiment of this invention, there is provided the aforementioned reagent, wherein either of R¹ and R⁶ is a light capturing moiety having a xanthene structure.

The present invention further provides the following protective group, method for removing the protective group, and compound having the protective group.

More specifically, the present invention provides a photodegradable protective group comprising 2-nitrobenzyl group bound with a light capturing moiety that can absorb light of around 470 nm or 3,5-dinitrobenzyl group bound with a light capturing moiety that can absorb light of around 470 nm. According to a preferred embodiment of this invention, there is provided the aforementioned protective group, which is for protecting hydroxy group or amino group.

The present invention also provides a method for removing a protective group comprising 2-nitrobenzyl group bound with a light capturing moiety that can absorb light of around 470 nm or 3,5-dinitrobenzyl group bound with a light capturing moiety that can absorb light of around 470 nm, which comprises irradiating a compound having the protective group with light of around 470 nm, and provides a compound having the aforementioned protective group.

From another aspect, the present invention provides a regent for introducing a photodegradable protective group, which is represented by the following formula (II) or (III): 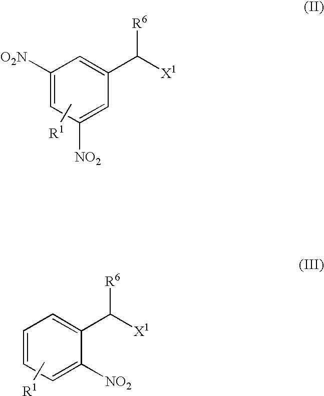
wherein X¹ represents a leaving group, R¹ represents hydrogen atom, or one to three arbitrary monovalent substituents substitutable on the benzene ring (when R¹ represents two or three substituents, they may be the same or different), and R⁶ represents hydrogen atom, one or two monovalent substituents, or one divalent substituent (when R⁶ represents two substituents, they may be the same or different), provided that at least one light capturing moiety that can absorb light of around 470 nm exists as R¹ or R⁶. As a preferred embodiment of this invention, there is provided the aforementioned reagent, wherein either of R¹ and R⁶ is a light capturing moiety having a xanthene structure.

### BRIEF EXPLANATION OF THE DRAWINGS

FIG. 1 shows changes in absorption spectra of 6-(3,5-dinitrobenzyloxy)-9-o-toluylxanthen-3-one and 6-(2-nitrobenzyloxy)-9-o-toluylxanthen-3-one, which were observed when they were irradiated with light of 470 nm.

FIG. 2 shows release of 6-hydroxy-9-o-toluylxanthen-3-one and resulting increase of fluorescence when 6-(3,5-dinitrobenzyloxy)-9-o-toluylxanthen-3-one was irradiated with light of 470 nm.

### BEST MODE FOR CARRYING OUT THE INVENTION

The photodegradable protective group of the present invention is characterized by comprising a benzyl group which is bound with a light capturing moiety that can absorb light of around 470 nm and having one or more nitro groups on the benzene ring. Although reactive functional group which can be protected with the protective group of the present invention is not particularly limited, examples include, for example, hydroxy group, amino group, carboxy group, phosphate group, and the like. Among them, hydroxyl group is preferred.

As the benzyl group bound with a light capturing moiety that can absorb light of around 470 nm and having one or more nitro groups on the benzene ring, 2-nitrobenzyl-group, and 3,5-dinitrobenzyl group are preferred.

The light capturing moiety that can absorb light of around 470 nm may be bound at an arbitrary substitutable position of the benzyl group having one or more nitro groups on the benzene ring. Although one or more of the light capturing moieties which can absorb light of around 470 nm may bind, it is preferred that one moiety binds. For example, it is desirable that one light capturing moiety that can absorb light of around 470 nm binds to a benzyl group having one or more nitro groups on the benzene ring at an arbitrary position on the benzene ring. Alternatively, when a compound to be protected itself can act as an light capturing moiety that can absorb the light of around 470 nm, a benzyl group having one or more nitro groups on the benzene ring may be bound to a reactive functional group such as hydroxy group and amino group of the compound to be protected. Such an embodiment also falls within the scope of the present invention. In such compounds, the benzyl group having one or more nitro groups on the benzene ring is cleaved by irradiation of visible light to regenerate the compound before the binding with the benzyl group having one or more nitro groups on the benzene ring.

When the light capturing moiety which absorbs the light of around 470 nm of the photodegradable protective group of the present invention absorbs light in the visible range (especially light of around 470 nm), the moiety causes photoinduced electron transfer with the benzyl moiety having nitro group on the benzene ring, and as a result, the photodegradable protective group is removed. Therefore, it is essential that the light capturing moiety absorbs light in the visible range (especially light of around 470 nm) so that photoinduced electron transfer is caused between the light capturing moiety, absorbing the light of a visible range (especially light of around 470 nm), and the benzyl moiety having nitro group on the benzene ring by irradiation of light in the visible range (especially light of around 470 nm). As the light capturing moiety which efficiently absorbs light in the visible range (especially light of around 470 nm) which causes the photoinduced electron transfer, those having intense fluorescence are preferred, with which deprotection of the photodegradable protective group can be efficiently performed.

As the light capturing moiety that can absorb light in the visible range (especially light of around 470 nm), light capturing moieties having a xanthene structure and the like are preferred. For example, a residue obtained by eliminating hydrogen atom from 6-hydroxy group of 6-hydroxy-9-o-toluylxanthen-3-one, and the like are more preferred. However, the light capturing moiety is not limited to these specific light capturing moieties. Residues obtained by suitably eliminating appropriate atoms such as hydrogen atom, hydroxy group, a halogen atom and the like from compounds having absorbing property of light in the visible range such as those having the fluorescein structure (mainly for light of around 450 to 490 nm), rhodamine structure (mainly for light of around 500 to 580 nm), or 4,4-difluoro-4-bora-3a,4a-diaza-s-indacene structure (BODIPY, registered trademark, 505/515, mainly for light of around 500 to 650 nm) can also be used as the light capturing moiety that can absorb light in the visible range in the same manner as described above.

As the reagent for introducing a benzyl group which is bound with a light capturing moiety that can absorb light in the visible range (especially light of around 470 nm) and having one or more nitro groups on the benzene ring, compounds represented by the aforementioned formula (I) can be used. In the formula, X¹ represents a leaving group, and groups generally used as a leaving group in the field of organic chemistry, for example, a halogen atom such as bromine atom and chlorine atom,

p-toluenesulfonyloxy group, methanesulfonyloxy group, and diazo group can be used. Although type of the monovalent substituent represented by R², R³, R⁴, R⁵, or R⁶ is not particularly limited, examples include, for example, a halogen atom (it may be any of fluorine atom, chlorine atom, bromine atom, and iodine atom), hydroxy group, amino group, carboxy group, sulfo group, an alkylsulfonate group, and the like. When R⁶ is constituted by a monovalent substituent, R⁶ represents one substituent, or the same or different two substituents substituting on the benzyl group to which R⁶ binds. Examples of the divalent substituent represented by R⁶ include, for example, an alkylenedioxy group, oxo group, and the like.

In the compounds represented by the aforementioned formula (I), at least one light capturing moiety that can absorb light in the visible range (especially light of around 470 nm) exists as R¹ or R⁶. Type of the light capturing moiety that can absorb light in the visible range (especially light of around 470 nm) is not particularly limited, and it can be suitably chosen by those skilled in the art as described above. When the aforementioned compounds are introduced as a protective group of a reactive functional group such as hydroxy group and amino group, a reaction may be performed according to a usual method for introducing a protective group by using an appropriate base or the like depending on the properties of the leaving group. As for the introduction of protective groups and deprotection, “Protective Groups in Organic Synthesis”, P. G. M. Wuts and T. Green, 3rd Edition, 1999, Wiley, John & Sons) and the like can be referred to.

As the compounds having the photodegradable protective group of the present invention, compounds consisting of a physiologically active substance and the like protected with the photodegradable protective group of the present invention are useful. Although original physiological activities of these compounds are completely lost before light irradiation, the original physiological activities can be temporally and spatially restored by light irradiation. For example, compounds consisting of a nucleotide having a physiological activity such as ATP, 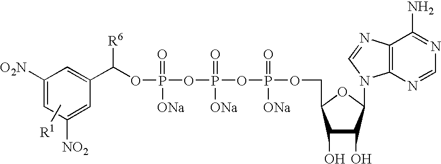
GTP and cAMP, an amino acid responsible for neurotransmission such as GABA, 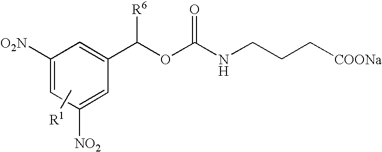
and glutamic acid, or the like, protected with the photodegradable protective group of the present invention can be used for researches of mechanisms of biological functions, and thus they are useful.

### EXAMPLES

The present invention will be more specifically explained with reference to examples. However, the scope of the present invention is not limited to the following examples.

### Example 1

### Synthesis of 6-(3,5-dinitrobenzyloxy)-9-o-toluylxanthen-3-one (3,5-DNB-2-Me TG)

6-Hydroxy-9-o-toluylxanthen-3-one (1.0 equivalent), and 3,5-dinitrobenzyl bromide (1.0 equivalent) were dissolved in N,N-dimethylformamide, and then cesium carbonate (1.0 equivalent) was added to this solution. The mixture was shielded from light with aluminum foil, and stirred overnight at room temperature under an argon atmosphere. After completion of the reaction, the reaction mixture was diluted with a huge volume of water, and extracted with ethyl acetate. After the solvent was evaporated, the residue was purified by using a silica gel column.

¹H-NMR (CDCl₃, 300 MHz) 2.09, 5.38, 6.46, 6.58, 6.88, 6.96, 7.03-7.06, 7.18, 7.35-7.48, 8.67, 9.06

HRMS (ESI+) calcd for C₂₇H₁₉N₂O₇: 483.1192 (M+H), found: 483.1184

### Example 2

### Synthesis of 6-(2-nitrobenzyloxy)-9-o-toluylxanthen-3-one (o-NB-2-Me TG)

In the same manner as that of Example 1, synthesis was performed by using 6-hydroxy-9-o-toluylxanthen-3-one (1.0 equivalent) and o-nitrobenzyl bromide (1.0 equivalent).

¹H-NMR (CDCl₃, 300 MHz) 2.08, 5.61, 6.46, 6.57, 6.86, 6.94, 7.02, 7.05, 7.16, 7.35-7.49, 7.55, 7.72, 7.84, 8.22

HRMS (ESI+) calcd for C₂₇H₂₀NO₅: 438.1342 (M+H), found: 438.1326

### Example 3

(a) Preparation of Sample

3,5-DNB-2-Me TG obtained in Example 1 and o-NB-2-Me TG obtained in Example 2 were each dissolved in 0.1 M sodium phosphate buffer (pH 7.4) at a concentration of 3 μM. As a cosolvent, 10% N,N-dimethylformamide was added.

(b) Light Irradiation

Light of 470 nm was irradiated by using an argon laser. One cycle consisted of 128 pulses×10 times.

(c) Results

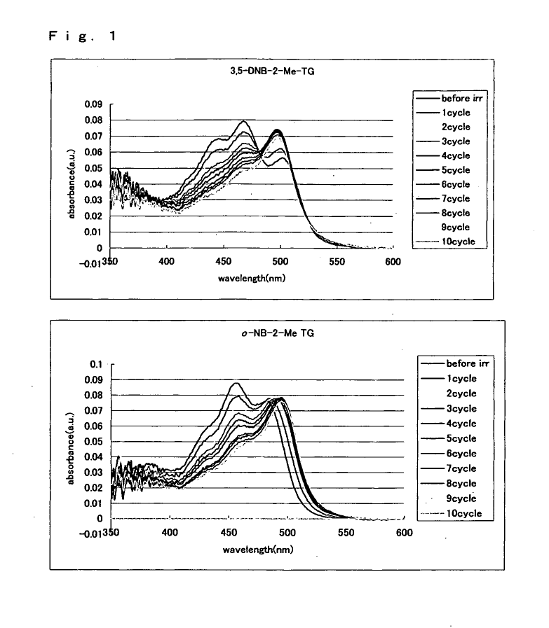

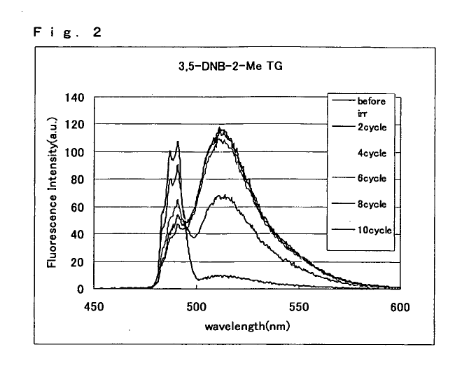

Changes of absorption spectra caused by the light irradiation are shown in FIG. 1. It can be understood that the peaks around 450 nm observed for 3,5-DNB-2-Me TG and o-NB-2-Me TG were lowered, and the peaks around 490 nm were highered with the light irradiation. This is because 6-hydroxy-9-o-toluylxanthen-3-one was released from the compounds by deprotection. From these results, it is clearly demonstrated that when a light capturing moiety, which can absorb visible light of 470 nm, binds to 3,5-dinitrobenzyl group or 2-nitrobenzyl group, the 3,5-dinitrobenzyl group or 2-nitrobenzyl group is easily removed by irradiation of visible light of 470 nm. FIG. 2 shows development of increase of fluorescence provided by 6-hydroxy-9-O— toluylxanthen-3-one observed when 3,5-DNB-2-Me TG was irradiated with light of 470 nm.

What is claimed is:

1. A photodegradable protective group comprising a benzyl group which is bound with a light capturing moiety that can absorb light of around 470 nm and has one or more nitro groups on the benzene ring.

2. The protective group according to claim 1, which is used for protecting hydroxy group or amino group.

3. A method for removing a protective group comprising a benzyl group which is bound with a light capturing moiety that can absorb light of around 470 nm and has one or more nitro groups on the benzene ring, which method comprises irradiating a compound having the protective group with light of around 470 nm.

4. A reagent for introducing a photodegradable protective group removable by irradiation of light of around 470 nm, which is represented by the following formula (I): 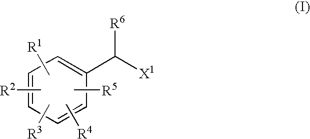
wherein X¹ represents a leaving group, R¹ represents hydrogen atom, or a light capturing moiety that can absorb light of around 470 nm; R², R³, R⁴, and R⁵ independently represent nitro group, hydrogen atom, or an arbitrary substituent substitutable on the benzene ring wherein when two to four of said groups represent substituents, they may be the same or different, provided that at least one of R², R³, R⁴, and R⁵ is nitro group; and R⁶ represents hydrogen atom, a light capturing moiety that can absorb light of around 470 nm, one or two monovalent substituents, or one divalent substituent wherein when the group represents two substituents, they may be the same or different, provided that at least one light capturing moiety that can absorb light of around 470 nm exists as R¹ or R⁶.

5. A photodegradable protective group comprising 2-nitrobenzyl group which is bound with a light capturing moiety that can absorb light of around 470 nm or 3,5-dinitrobenzyl group which is bound with a light capturing moiety that can absorb light of around 470 nm.

6. The protective group according to claim 5, which is used for protecting hydroxy group or amino group.

7. A method for removing a protective group comprising 2-nitrobenzyl group which is bound with a light capturing moiety that can absorb light of around 470 nm or 3,5-dinitrobenzyl group which is bound with a light capturing moiety that can absorb light of around 470 nm, which method comprises irradiating a compound having the protective group with light of around 470 nm.

8. A regent for introducing a photodegradable protective group removable by irradiation of light of around 470 nm, which is represented by the following formula (II) or (III): 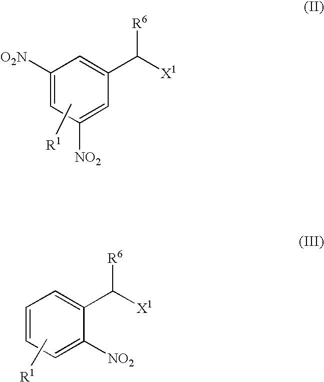
wherein X¹ represents a leaving group; R¹ represents hydrogen atom, or one to three arbitrary monovalent substituents substitutable on the benzene ring wherein when the group represents two or three substituents, they may be the same or different; and R⁶ represents hydrogen atom, one or two monovalent substituents, or one divalent substituent wherein when the group represents two substituents, they may be the same or different, provided that at least one light capturing moiety that can absorb light of around 470 nm exists as R¹ or R⁶.
